(19) United States
(12) Patent Application Publication
(10) Pub. No.: US 2006/0224160 A1
(43) Pub. Date: Oct. 5, 2006

(54) Instruments and methods for aggressive yet continuous tissue removal

(76) Inventors: Hai H. Trieu, Cordova, TN (US); Lehmann K. Li, Milford, CT (US); Michael C. Sherman, Memphis, TN (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 11/095,652

(22) Filed: Apr. 1, 2005

(51) Int. Cl. A61B 17/00

(52) U.S. Cl. 606/83

(57) ABSTRACT

Devices and methods for aggressively and continuously cutting and removing anatomical tissue. The device may comprise elongated outer and inner tubes having respective proximal and distal ends. The inner tube is moveably received in the elongated inner. A cutting assembly is rotatably received in the elongated inner tube. The device may also comprise a handle arm attached near the proximal end of the outer tube, and a trigger arm attached near the proximal end of the inner tube. A movable jaw is pivotally attached near the distal end of the outer or inner tube, the movable jaw being urged towards the cutting blade assembly as the trigger arm is actuated.

### FIELD OF THE INVENTION

Embodiments of the systems and methods described herein relate to devices and methods for cutting anatomical tissue, and more particularly to devices and methods for aggressively and continuously cutting tissue in preparation for intervertebral surgery.

### DESCRIPTION OF THE RELATED ART

The intervertebral disc functions to stabilize the spine and to distribute forces between vertebral bodies. The intervertebral disc is composed primarily of three structures: the nucleus pulposus, the annulus fibrosis, and two vertebral end-plates. The nucleus pulposus is an amorphous hydrogel in the center of the intervertebral disc. The annulus fibrosis, which is composed of highly structured collagen fibers, maintains the nucleus pulposus within the center of the intervertebral disc. The vertebral end-plates, composed of hyalin cartilage, separate the disc from adjacent vertebral bodies and act as a transition zone between the hard vertebral bodies and the soft disc.

Intervertebral discs may be displaced or damaged due to trauma, disease, or the normal aging process. One way to treat a displaced or damaged intervertebral is by surgical removal and replacement of all or a portion of the intervertebral disc, including the nucleus and the annulus fibrosis. When the entire intervertebral disc is to be replaced, both the nucleus and the annulus fibrosis must be removed. Such a procedure requires an aggressive cutting tool that is able to cut through tissue of varying hardness and toughness commonly associated with the annulus fibrosis. However, while hard or tough tissue is not commonly encountered when only the nucleus is being removed and replaced, particular care must be taken so that the annulus fibrosis (which surrounds the nucleus) is not severely damaged during the tissue removal process. Thus, it is desirable in such a situation to have a device that provides continuous cutting and removal of tissue so that damage to the annulus fibrosis is minimized.

Numerous devices currently exist for removing all or a portion of an intervertebral disc. One such device is the pituitary or manual rongeur. The pituitary rongeur is a manually-operated device comprising an elongated shaft having a jaw-like member at the distal end of the shaft that may be used to grab and remove disc tissue. On the proximal end of the shaft is a trigger mechanism that is used to actuate the jaw. The pituitary rongeur enables its user to aggressively cut and remove tissue, regardless of hardness or toughness, and provides the user with the benefit tactile sensation. However, pituitary rongeurs require that the tool be periodically removed from the area or site in order to dispose of cut tissue. Thus, while pituitary rongeurs are particularly valuable for complete disc replacement procedures (i.e., both the nucleus and annulus fibrosis are being removed), they are not as desirable when only the nucleus is to be removed and replaced.

Another device better suited for this purpose is the rotary cutter or shaver. The rotary cutter or shaver typically comprises an outer member and an inner member that is rotatably received in the outer member. The inner member may further comprise a cutting member that alone or in cooperation with the outer member functions to cut tissue. A rotary cutter or shaver may further comprise a power source that drives the cutting action, and a suction source that aspirates and removes cut tissue through a suction passageway within the cutter or shaver. Rotary cutters or shavers may be straight or angled to facilitate the cutting and removal process, and may include a network of blades or a screw-type element to effect the cutting action. While rotary cutters and shavers are able to continuously cut and remove tissue without having to be physically removed from the cutting area or site (e.g., intervertebral space), they suffer from the inability to cut tissue of greater hardness or toughness.

Exemplary rotary cutters and shavers are described in the following U.S. patents:

U.S. Pat. No. 6,656,195 discloses a flexible inner tubular member for being rotated in an outer tubular member of a rotary tissue cutting instrument. The flexible tubular member includes an elongate inner tube having a rotatably driveable proximal end, a distal end having a cutting member exposed from the outer tubular member to cut anatomical tissue and a flexible region allowing the inner tubular member to rotate within the outer tubular member while conforming to the shape of the outer member. The flexible region is defined by a helical cut in the inner tube defining a plurality of integrally, unitarily connected tube segments angularly movable relative to one another and a spiral wrap disposed over the helical cut.

U.S. Pat. No. 6,620,180 discloses a laryngeal cutting blade that has an outer blade assembly including a tubular member having a bend closer to a proximal portion than a distal end. The cutting blade also includes a hollow inner blade assembly rotatably received in the outer blade assembly for mounting to a powered handpiece and having a flexible portion disposed adjacent the bend and a suction passage extending through the inner blade assembly to permit aspiration of cut tissue, the angled configuration of the laryngeal cutting blade and toothed and smooth profile cutting ends therefore are particularly effective for debulking large, firm lesions and for delicate shaving of superficial lesions of the vocal cords after removal of bulk mass.

U.S. Pat. No. 6,533,749 discloses an angled rotary tissue cutting instrument that includes an outer member or blade. The cutting instrument has a rigid outer tube with proximal and distal portions connected by a bend, and an inner member or blade rotatably disposed within the outer member and including an inner tube of integral one-piece construction having a spiral cut formed therein between proximal and distal ends thereof to define a flexible region adjacent the bend, and at least one layer of a spirally wound strip of material superimposed over the spiral cut portion of the inner tube. The spiral cut preferably extends to a cutting tip at the distal end of the inner tube so that the inner member can be bent closer to the distal end thereof to access difficult to reach areas of the head and neck, and other parts of the body.

U.S. Patent No. RE38,018 discloses an angled rotary tissue cutting instrument including an outer blade assembly, having a rigid tubular member with proximal and distal portions connected by a bead, and an inner blade assembly rotatably disposed within the outer blade assembly and including a tubular drive shaft at a proximal end, a cutting tip at a distal end, and a flexible coupling disposed between the drive shaft and the cutting tip. The drive shaft and cutting tip include neck portions which are disposed telescopically within proximal and distal ends of the coupling. The flexible coupling is formed of a flexible polymeric material, and each of the neck portions includes a lateral opening defining a predetermined flow path for the polymeric material during fabrication so that the flexible polymeric coupling includes flow portions extending into the openings in the neck portions of the drive shaft and the cutting tip to form permanent, interlocking mechanical joints therewith capable of receiving and transmitting torque.

U.S. Pat. No. 6,183,433 discloses a surgical suction cutting instrument that includes a tubular outer member defining a cutting chamber with an opening, an inner member with a distal cutting edge movably received in the outer tubular member and a flushing mechanism for supplying fluid to the cutting chamber via an outlet communicating with the cutting chamber. The cutting edge of the inner member is disposed within the cutting chamber adjacent the opening to engage bodily tissue through the opening, and a lumen is preferably defined through the inner tubular member in communication with the cutting chamber for aspirating cut bodily tissue. In one embodiment, the flushing mechanism includes a tubular member disposed alongside the outer tubular member and having an outlet communicating with the cutting chamber for supplying fluid to the cutting chamber to prevent and clear clogging of the passage without the need of having to remove the cutting instrument from the surgical site.

Commercial rotary cutter or shaver systems, also known as power tissue resectors, are available under the various brand names, such as the StraightShot™ Magnum II™ Microsector™ System™ or the MSD Pyrametrix Plus™, for example. Such systems may use a blade-based cutter or shaver, such as a RAD™ Straight or Curved Sinus Blade, for example, or a screw-type cutter. Other types or forms of cutters or shavers may of course be used.

Currently, however, no tissue-cutting device, tool or system exists that exhibits the aggressive and tactile sensory characteristics of the pituitary rongeurs along with the continuous cutting and removal capability of existing rotary cutters or shavers. During preparations for a disc replacement procedure, for example, a device having these characteristics would enable its user to more efficiently cut and remove tissue without having to periodically remove the device from the intervertebral space to dispose of cut tissue. Similarly, such a device would enable its user to continuously cut and remove tough or hardened tissue that current rotary cutters or shavers are unable to cut.

The description herein of problems and disadvantages of known devices, methods, and apparatuses is not intended to limit the systems and methods described herein to the exclusion of these known entities. Indeed, embodiments of the systems and methods described herein may include one or more of the known devices, methods, and apparatuses without suffering from the disadvantages and problems noted herein.

### BRIEF SUMMARY OF THE INVENTION

What is needed are devices and methods for cutting and removing anatomical tissue in an aggressive and continuous manner. Additionally, there is a need for a cutting device that provides its user with the tactile sensation and aggressiveness afforded by existing pituitary rongeurs, and that simultaneously provides the continuous cutting and removal action afforded by existing rotary cutters and shavers. Additionally, there is a need for a cutting device that is simple and convenient to use. There is also a need for a cutting device that permits active mechanical grabbing of tissue, while providing automatic removal of cut tissue, such as through a suction tube or passageway, for example. Embodiments of the systems and methods described herein solve some or all of these needs, as well as additional needs.

Therefore, in accordance with an embodiment of the systems and methods described herein, there is provided a tissue cutting tool. The tool may comprise an elongated outer tube having a proximal end and a distal end, and an elongated inner tube having a proximal end and a distal end. The elongated inner tube may be moveably received in the elongated outer tube. The tool may also comprise a cutting assembly that may be received in the elongated inner tube. The tool may also comprise a handle arm attached near the proximal end of the outer tube, and a trigger arm attached near the proximal end of the inner tube. The tool may also comprise a movable jaw pivotally attached near the distal end of the outer or inner tube. The movable jaw may be urged towards a cutting portion of the cutting blade assembly as the trigger arm is actuated.

In accordance with another embodiment of the systems and methods described herein, an anatomical tissue cutting tool is provided. The cutting tool may comprise a bent rigid outer tube having a proximal end and a distal end; a flexible inner tube having a proximal end and a distal end, the elongated inner tube being moveably received in the elongated outer tube; a cutting blade assembly being received in the flexible inner tube; a handle arm attached near the proximal end of the outer tube; a trigger arm attached near the proximal end of the inner tube; and a movable jaw pivotally attached near the distal end of the outer or inner tube, the movable jaw being urged towards a cutting portion of the cutting blade assembly as the trigger arm is actuated.

In accordance with another embodiment of the systems and methods described herein, there is provided a flexible inner member assembly for being moved in a bent outer member assembly. The flexible inner member comprises a flexible member defining an inner lumen for receiving a cutting assembly. The flexible inner member may also comprise a jaw or arm member pivotally attached to or near the distal end of the flexible member. The jaw or arm member may be actuated by a trigger member associated with the bent outer tubular member. During actuation, the jaw or arm member may be urged towards the cutting assembly.

In accordance with another embodiment of the systems and methods described herein, there is provided a tissue cutter. The tissue cutter comprises a continuous cutting assembly associated with or near a distal end of a cutter handle assembly; and a mechanical gathering assembly attached to or near the distal end of the cutter assembly.

In accordance with another embodiment of the systems and methods described herein, there is provided a method for cutting tissue. The method comprises the steps of entering an area containing tissue, particles, debris or foreign material with any of the cutters described herein; and using the cutter to aggressively and continuously cut tissue, particles debris, or foreign material from within the area.

These and other features and advantages of the systems and methods described herein will be apparent from the description provide herein.

### BRIEF DESCRIPTION OF THE DRAWINGS

FIG. 1 is an illustration of a cutting system using an exemplary device according to embodiments of the systems and methods described herein.

FIG. 2 is an illustration of an exemplary device according to embodiments of the systems and methods described herein.

FIG. 2a-2i are illustrations of embodiments of cutting assemblies that may be used with the systems and methods described herein.

FIG. 2j-2k are illustrations of embodiments of cutting assemblies that may be used with the systems and methods described herein.

FIG. 3 is an illustration of an exemplary device in an opened position according to embodiments of the systems and methods described herein.

FIG. 4 is an illustration of an exemplary device in a closed position according to embodiments of the systems and methods described herein.

FIGS. 4a and 4b illustrate exemplary devices for cutting tissue according to embodiments of the systems and methods described herein.

FIG. 5 is an illustration of a jaw member according to embodiments of the systems and methods described herein.

FIG. 6 is an illustration of a link member according to embodiments of the systems and methods described herein.

FIG. 7 is an illustration of an exemplary device in an opened position according to embodiments of the systems and methods described herein.

FIG. 7a is an illustration of the interrelationship between a jaw member and a ball pivot according to embodiments of the systems and methods described herein.

FIG. 8 is an illustration of an exemplary device in a closed position according to embodiments of the systems and methods described herein.

FIG. 8a is an illustration of the interrelationship between inner and outer members, a jaw member, and a ball pivot according to embodiments of the systems and methods described herein.

FIG. 9 is a top view illustration of an outer tube assembly according to embodiments of the systems and methods described herein.

FIG. 10 is a top view illustration of an inner tube assembly according to embodiments of the systems and methods described herein.

FIG. 11 is a perspective view illustration of an outer tube assembly according to embodiments of the systems and methods described herein.

FIG. 12 is a perspective view of an inner tube assembly according to embodiments of the systems and methods described herein.

FIG. 13 is a side view of a ball pivot according to embodiments of the systems and methods described herein.

FIG. 14 is a perspective view of a jaw member according to embodiments of the systems and methods described herein.

FIG. 15 is an illustration of a straight tip configuration of a cutting tool according to embodiments of the systems and methods described herein.

FIG. 16 is an illustration of a positive angled or curved tip of a cutting tool according to embodiments of the systems and methods described herein.

FIG. 17 is an illustration of a negative angled or curved tip of a cutting tool according to embodiments of the systems and methods described herein.

FIG. 18 is an illustration of a positive angled or curved tip of a cutting tool having a aft-grabbing jaw according to embodiments of the systems and methods described herein.

FIGS. 19-22 illustrate various methods according to embodiments of the systems and methods described herein.

### DETAILED DESCRIPTION OF THE INVENTION

The following description is intended to convey a thorough understanding of the various embodiments of the systems and methods described herein by providing a number of specific embodiments and details involving devices and methods for cutting tissue within an intervertebral disc space. It is understood, however, that the systems and methods described herein is not limited to these specific embodiments and details, which are exemplary only. It is further understood that one possessing ordinary skill in the art, in light of known systems and methods, would appreciate the use of the systems and methods described herein for its intended purposes and benefits in any number of alternative embodiments.

Throughout this description, the expressions “intervertebral space” and “intervertebral disc space” refer to any volume or void between two adjacent vertebrae. The intervertebral disc space may be the volume inside of the annulus fibrosis of the intervertebral disc. Alternatively, the intervertebral disc space also may include the annulus fibrosis itself. The intervertebral disc space also may include either a portion or the entire volume inside the annulus fibrosis.

It is a feature of an embodiment of the systems and methods described herein to provide an instrument that aggressively and continuously cuts and/or removes tissue within the intervertebral space. It is a further feature of the systems and methods described herein to provide an instrument that enables its user to experience the tactile sensation and aggressiveness afforded by existing pituitary rongeurs, and that simultaneously provides the continuous cutting and removal action afforded by existing rotary cutters and shavers. Devices according to embodiments of the systems and methods described herein may, among other things, be useful to prepare an intervertebral space to receive a nucleus replacement or a full disc implant.

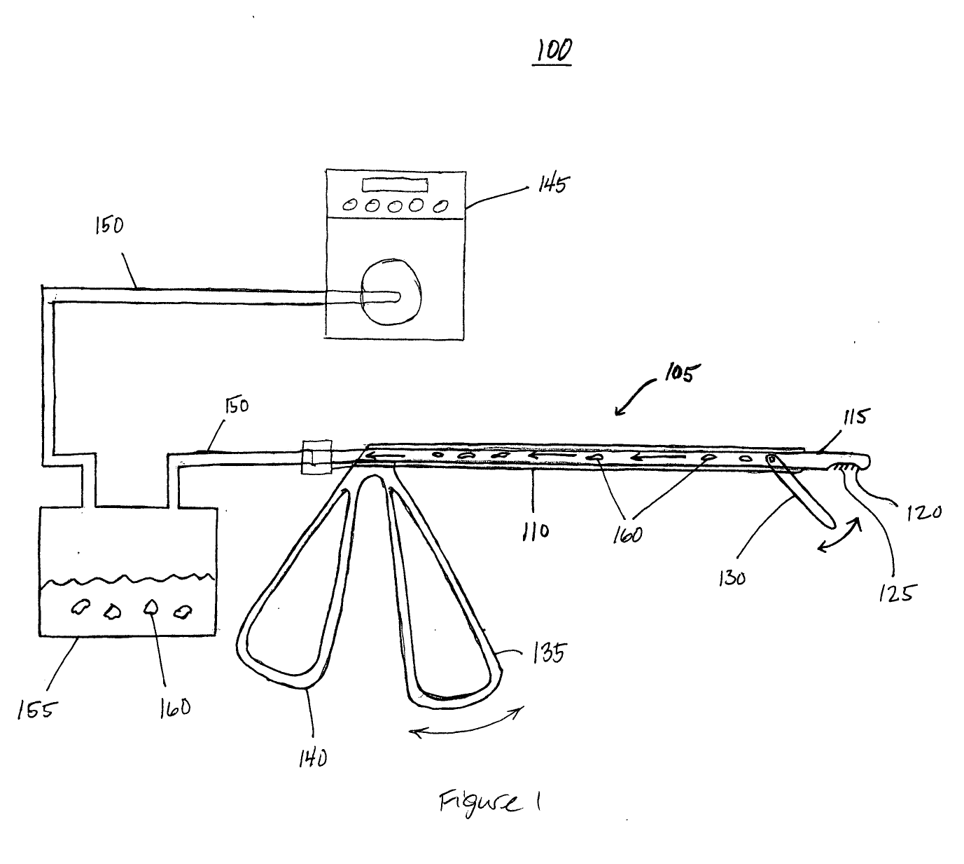

FIG. 1 depicts a system 100 for cutting tissue within the intervertebral disc space according to embodiments of the systems and methods described herein. As shown, system 100 comprises a cutting tool 105 connected to a vacuum pump or source 145 via tubing 150 and collection tank 160. Cutting tool 105 may, according to some embodiments of the systems and methods described herein, comprise an outer portion 110, an inner portion 115, a cutting window 120 at or near the distal end of the inner portion 115, a jaw or arm 130, a trigger 135 and a handle 140. In some embodiments, cutting tool 105 may further comprise a cutting assembly 125 inserted within inner portion 115. Cutting assembly 125 may comprise, for example, any of the devices described in U.S. Pat. No. 6,656,195 to Peters, et al.; U.S. Pat. No. 6,620,180 to Bays, et al.; U.S. Pat. No. 6,533,749 to Mitusina et al.; U.S. Patent No. RE38,018; and U.S. Pat. No. 6,183,433 to Bays. The specification and drawings of each of these patents is specifically incorporated by reference herein. In some embodiments, cutting assembly 125 may comprise any rotary or non-rotary cutting device that is able to cooperate with tool 105 to cut and/or remove tissue. Other cutting assemblies may be used.

Cutting tool 105 may also comprise a jaw or arm 130 that acts to urge or guide tissue material towards window 120 and cutting assembly 125. In some embodiments, a user of tool 105 may selectively position jaw or arm 130 by actuating trigger 135. That is, as trigger 135 is squeezed towards handle 140, for example, jaw or arm 130 may close towards window 120 and assembly 125. This way, tissue or loose particles that may be contained in the site being worked on may be forced against cutting assembly 125 to enhance the cutting and removal action. Similarly, jaw or arm 130 may be used to aggressively grab and cut hard or tough tissue that otherwise would not get cut by cutting assembly 125. Accordingly, tool 105 provides the user with the aggressive and tactile sensing capabilities of the pituitary or manual rongeur and the continuous cutting and removal features of existing power driven cutters and shavers, for example.

As shown in FIG. 1, tissue cut by tool 105 may be automatically removed through an internal suction passageway of the cutting assembly 125. In some embodiments, such an internal passageway may be connected to a vacuum pump or source 145 via tubing 150 and collection tank 160. Accordingly, tissue cut by tool 105 may be automatically removed and disposed of.

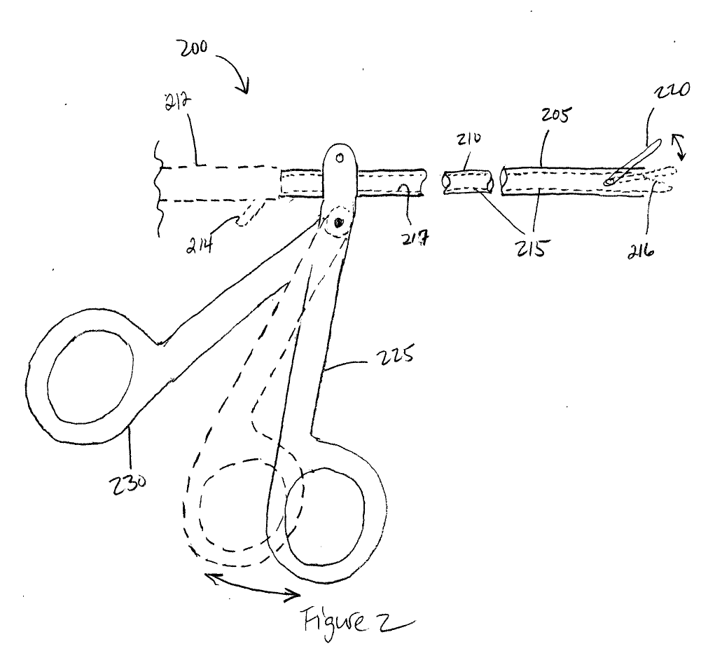

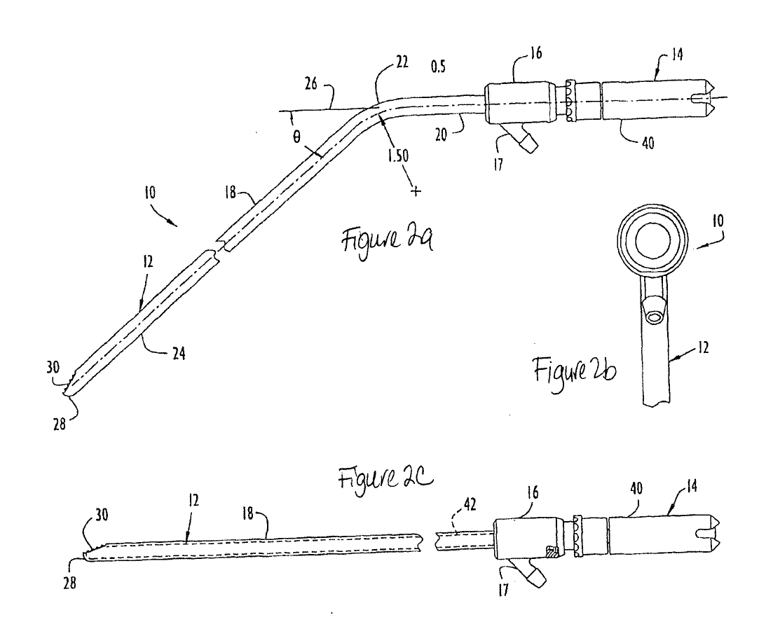

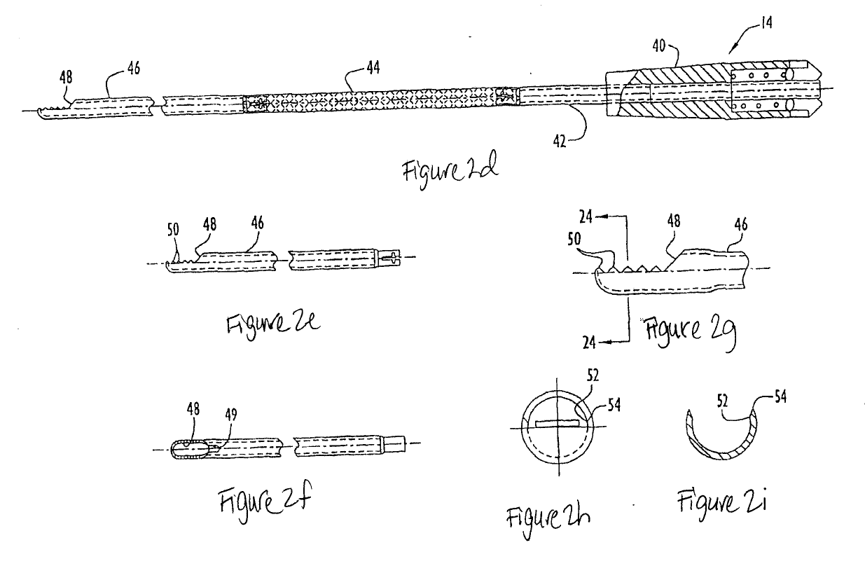

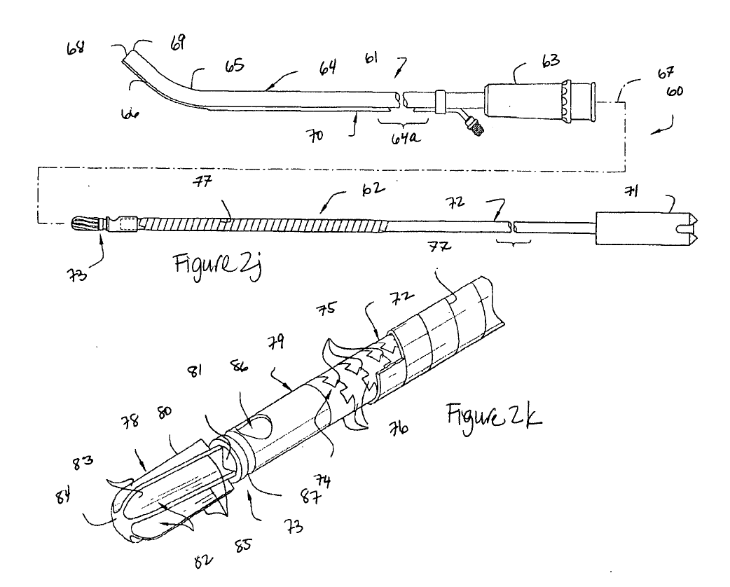

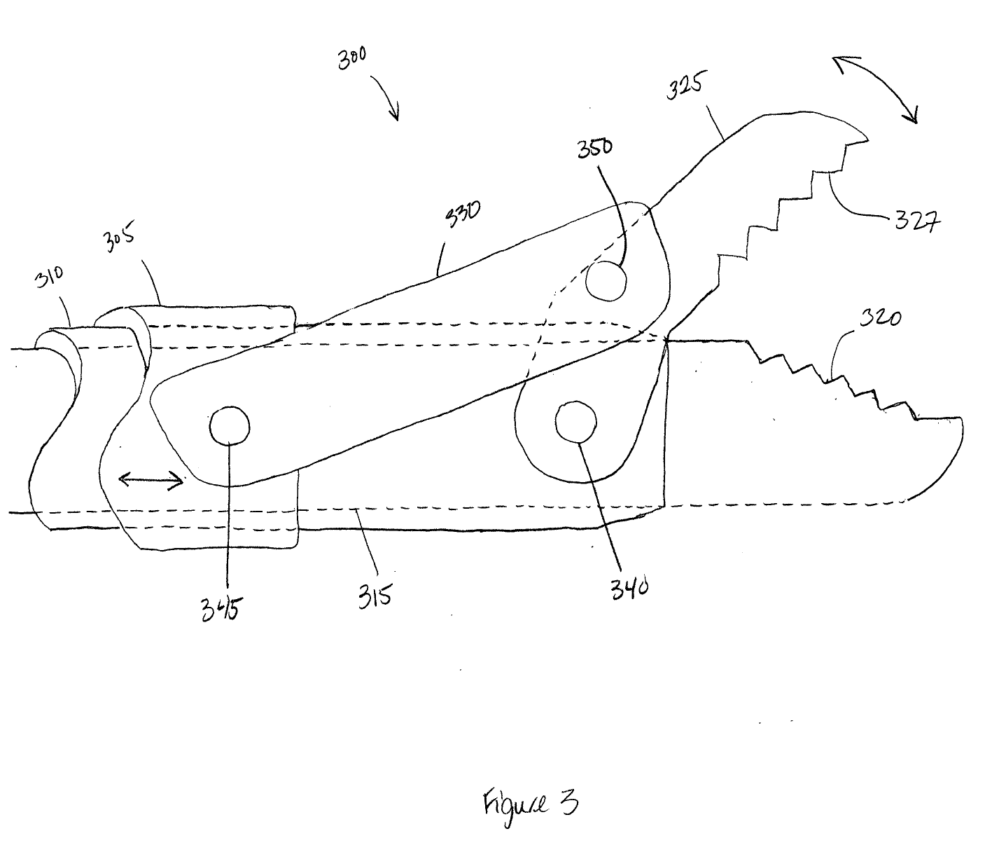

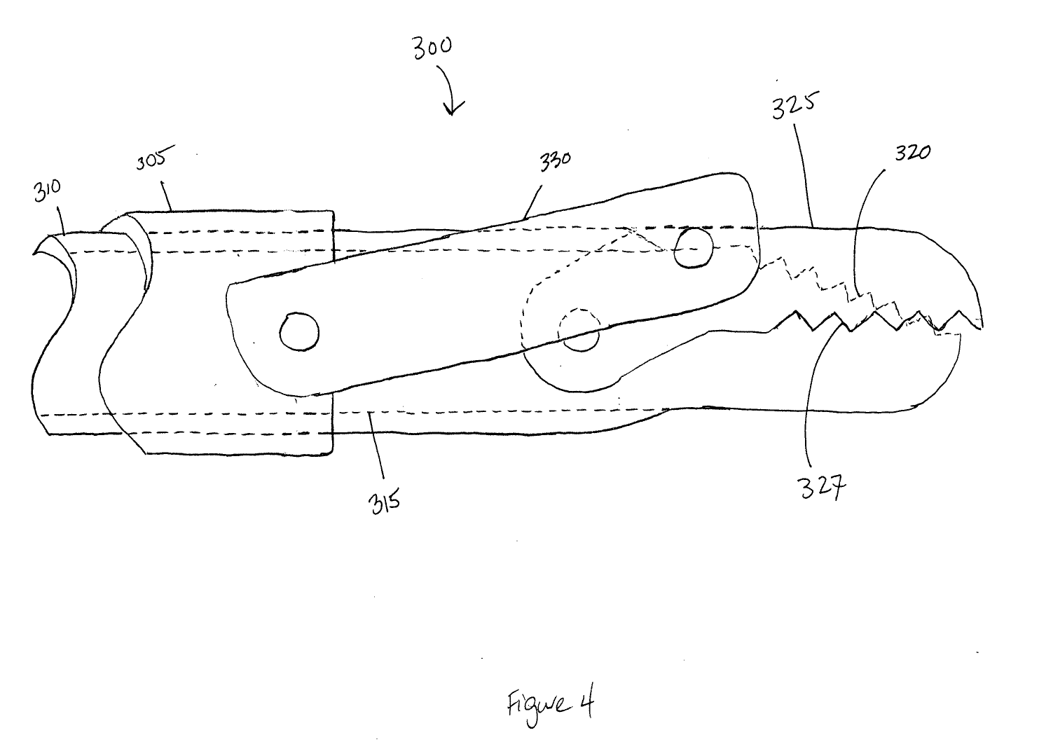

FIG. 2 depicts a more detailed illustration of a cutting tool 200. As shown, tool 200 comprises an outer tubular member 205, an inner tubular member 210, jaw or arm 220, trigger 225, and handle 230. Inner tubular member 210 is movably received within outer tubular member 205. For intervertebral use, the outer tubular member 205 may have a diameter of smaller than about 10 millimeters, preferably smaller than about 8 millimeters, and most preferably smaller than about 6 millimeters. For other uses, the preferred diameter of each tubular member may depend on the area being treated and/or the size of tissue particles or other material expected to be removed. As explained above, the user may actuate the jaw or arm 220 by squeezing trigger 225. For example, if trigger 225 is at its rightmost position as shown in FIG. 2, jaw or arm 220 may be in a completely opened positioned. When trigger 225 is fully squeezed, as shown by the dashed trigger, jaw or arm 220 may by in a fully closed position. In some embodiments, the precise coordination between trigger position and jaw or arm position may be arranged as necessary. Thus, a fully squeezed trigger, for example, may result in the jaw or arm being partly opened. As will be explained below, the interaction between trigger and jaw or arm may result from the relative positioning of inner and outer tubular members 205 and 210.

In some embodiments, tool 105 may further include a cutting assembly 215. Cutting assembly 215 may be rotatably received within inner tubular member 210, as shown in FIG. 2. Cutting assembly 215 may include a cutter portion 216 that serves to continuously cut tissue. As will be described below, cutter portion 216 may comprise a power-driven rotary blade or screw-type assembly that continuously asserts a cutting action on tissue. In some embodiments, the rotary speed of cutter portion 216 may be controlled by a user of tool 105. Accordingly, the user may increase the rotary speed for harder or tougher tissues, and reduce it for softer tissues.

Cutting assembly 215 may also comprise a hub member 212 that serves to connect cutting assembly 215 to a power source and/or a vacuum or suction source and associated tubing. The vacuum or suction source may facilitate the removal cut tissue through internal passageway 217 of cutting assembly 215. For example, as tissue is cut by cutter portion 216, tissue debris may be removed (e.g. aspirated) through passageway 217 and out of tool 200 through hub 212 and into an associated collection tank, for example. Hub 212 may include an irrigation port 214 that may connect to any source of saline solution, for example, that cleanses the internal passageway 216 of cutting assembly 215. Such saline solution may also facilitate the removal of cut tissue by further flushing such tissue through passageway 217. In some embodiments, irrigating the passageway may provide lubrication throughout the inner walls of passageway 217 to further enhance the removal process.

As described above, tool 200 may be used to prepare an intervertebral space for surgery by aggressively and continuously cutting unwanted tissue. For example, the user may use the tactile sensory capability of the trigger and jaw or arm to detect and grab or tear tough or hard tissue. The user may also actuate the trigger to urge particles, debris or tissue towards cutting portion 216 for eventual removal. For example, such particles, debris or cut tissue can then be automatically removed (via suction or flushing) without having to remove the tool from the area or site risking damage to the surrounding tissue, such as the annulus fibrosis, for example. Accordingly, in the context of intervertebral tissue, tool 105 is appropriate for removing both soft nucleus tissue as well as harder or tougher annulus tissue. Moreover, tool 105 reduces the damage to annulus tissue defects during a nucleotomy by reducing instrument insertions and removals.

Various cutting assemblies are currently available that may be used with the systems and methods described herein. FIGS. 2a-2i, for example, illustrate a blade-type cutting assembly disclosed by U.S. Pat. No. 6,620,180. FIGS. 2j and 2k disclose a screw-type cutting assembly disclosed by U.S. Pat. No. 6,656,195. Other cutting assemblies may of course be used. Preferably, each cutting assembly is received within the inner members of the various cutting tools described herein.

FIGS. 2a-2c illustrate a powered cutting blade 10 that may be used with the systems and methods described herein and can be driven by any suitable handpiece such as the STRAIGHTSHOT™ marketed by Xomed Surgical Products, Inc. and shown in U.S. Pat. No. 5,916,231 to Bays, the disclosure of which is incorporated herein by reference. The blade 10 includes an outer blade member or assembly 12 and a hollow inner blade member assembly 14 rotatably received within the outer blade member. Outer blade member 12 includes a hub 16 with an irrigation port or coupling 17 extending angularly therefrom and an outer tubular member or sleeve 18 having a proximal portion 20 of straight configuration extending distally from the hub to a proximal bend 22 connecting the proximal portion with a distal portion 24 oriented at an angle .theta. of about 45.degree. relative to the longitudinal axis 26 of the proximal portion. The proximal bend 22 is closer to the proximal portion 20 than to the distal end 28 to provide desired spacing of the distal end from the proximal portion. Angled straight distal portion 24 of the outer tubular member extends downwardly from bend 22, looking at FIG. 2a, a distance greater than the distance between bend 22 and the proximal portion and portion 24 extends to a rounded distal end 28 having an opening facing upwardly, away from the center of curvature of the bend, to define a cutting port or window 30. Dependent upon the anatomical situation and requirements, the window 30 can face in any desired direction (i.e. also downwardly or sidewards) and the outer blade member 12 can have multiple curves or bends, such as 22, along the length thereof.

FIGS. 2d-2i illustrate an inner assembly 14 for the blade according to the systems and methods described herein, the inner assembly including a hub 40, a drive shaft 42 extending distally from the hub to a flexible coupling 44, and a cutting tip or cutter 46 extending distally from the flexible coupling. Cutting tip 46 includes a distal opening 48 defined by a peripheral edge with teeth 50 having a blunt inner surface or edge 52 and a sharp outer surface or edge 54. The distal opening communicates with a lumen defined by the inner assembly to permit tissue evacuation when the blade is connected with a source of suction. The inner assembly 14 has a size to rotate within the outer blade member 14 with sufficient annular space for irrigating fluid to pass between the inner and outer members from port 17 to the cutting tip via a flat 49 adjacent and aligned with opening 48 in order to prevent clogging of cut tissue in the lumen of the inner blade. The cut tissue is aspirated from the surgical site in a straight path through hub 40 to also prevent clogging and can be drawn through a STRAIGHTSHOT handpiece in a straight path.

FIG. 2j illustrates a rotary screw-type tissue cutting instrument 60 that may be used with the systems and methods described herein. As illustrated in FIG. 2j, instrument 60 includes an outer tubular member 61 and a flexible inner tubular member or blade 62 rotatably received within the outer member. Outer member 61 includes an outer hub 63 and a rigid outer tube or shaft 64 having a proximal length portion 64a of longitudinally or axially straight configuration extending distally from the hub to a bend, angle or curve 65 connecting the proximal length portion with a distal length portion 66 of longitudinally or axially straight configuration oriented at an angle relative to a central longitudinal axis 67 of the proximal length portion. Distal length portion 66 extends upwardly from bend 65, looking at FIG. 2j, to an open distal end 68 defining an opening circumscribed by a peripheral or circumferential edge. A curved recess, notch or indentation 69 may be formed in outer tube 64 along the circumferential edge, the recess 69 extending in a proximal direction and being disposed at the top of the outer tube when the outer member is oriented as shown in FIG. 2j. The outer member may be the same or similar to the outer member disclosed in prior application Ser. No. 09/495,350 filed Feb. 1, 2000, the disclosure of which is incorporated herein by reference. Accordingly, the outer member may include an irrigation passage 70 as disclosed in the aforementioned application. The radius of curvature for the bend, the size of the bend angle, the location of the bend relative to the distal end of the outer member, and the direction of the bend are dependent upon the procedure to be performed and the location of an operative site or area to be accessed. For example, the outer member can have any of the configurations described in U.S. patent application Ser. No. 09/404,461 filed on Sep. 24, 1999 and Ser. No. 09/074,739 filed on May 8, 1998 and now U.S. Pat. No. 5,922,003, the disclosures of which are incorporated herein by reference.

As shown in FIG. 2j, inner member 62 includes an inner hub 71 disposed proximally of the outer hub when the inner member is disposed within the outer member, an elongate inner tube or shaft 72 extending distally from the inner hub to be disposed coaxially or concentrically within the outer tube, and a cutting member 73 disposed at a distal end of the inner tube. Inner tube 72 is formed from a rigid tube made of medically acceptable material such as stainless steel. Tube 72 has a hollow cylindrical configuration with a cylindrical wall defining a lumen entirely through tube 72. As best seen in FIG. 2k, a helical or spiral cut 74 is formed through the cylindrical wall of tube 72. The helical cut 74 has a plurality of angled cut segments 75 on opposing sides of the tube 72, the angled cut segments 75 for the left side of tube 72, looking distally, being shown in FIG. 2k. The angled cut segments 75 are axially or longitudinally spaced from one another lengthwise along the tube 72 to form a plurality of serially arranged, interconnected helical or spiral tube segments 76 between the cutting member 73 and a proximal end of the inner tube 72.

The helical cut 74 is preferably formed in rigid tube 73 by laser cutting. The helical cut 74 extends continuously in a helical or spiral path, i.e. an open path, along the cylindrical wall forming tube 72 and about a central longitudinal axis of tube 72, such that opposite ends of the helical cut do not meet. The helical cut 74 extends in the radial direction through the entire thickness of the cylindrical wall so that each angled cut segment 75 is disposed between a pair of adjacent tube segments 76. Since the helical cut 74 extends continuously in the helical or spiral path, the tube segments 76 are materially or physically connected or joined to one another, with adjacent tube segments being integrally, unitarily connected in a helical or spiral fashion. The helix angle alpha (not shown) for helical cut 74 results in the angled cut segments 75 being disposed at angle a relative to the central longitudinal axis of tube 72. The helical cut 74 extends around the central longitudinal axis of the inner tube in a first direction, the helical cut 74 extending about the central longitudinal axis of inner tube 72 with a clockwise or right hand turn or slant looking from distal to proximal, such that the angled cut segments 75 on the left side of tube 72 extend proximally with a downward slant at angle a relative to the central longitudinal axis of tube 72. Of course, it should be appreciated that the angled cut segments on the right side of tube 72 extend distally with a downward slant at angle a relative to the central longitudinal axis of tube 72.

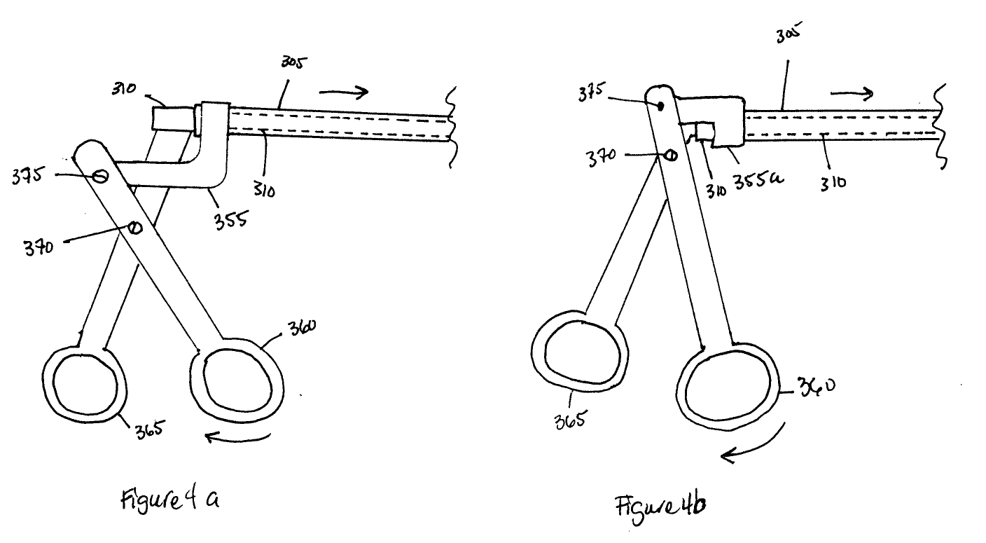

FIG. 3 illustrates a cutting tool 300 with a forward-grabbing jaw member according to various embodiments of the systems and methods described herein. As shown, tool 300 comprises an outer tubular member 305, an inner tubular member 310 movably received within member 305, a jaw member 325 with teeth 327, a link 300, and a cutting assembly 315 with cutting portion 320. Preferably, outer and inner tubular members comprise rigid hollow and elongated tubes. In some embodiments, jaw member 325 is pivotally attached to inner tubular member 310 via pin 340. Jaw member 325 may also be pivotally attached to outer member 305 via link 330. Link 330 is attached to outer member 305 at pin 345 and to jaw member 325 at pin 350. In this arrangement, actuation (e.g. squeezing) of the trigger member (not shown) associated with tool 300 may result in outer member 305 moving in a lateral direction relative to inner tubular member 310. Thus, when the trigger is actuated, outer member 305 may move rightward relative to inner tubular member 310 (see FIGS. 4a and 4b for tool arrangements that apply the requisite forces). Such action results in a force being transferred through link 330 to a pin 350 that translated to a torque force about pin 340 that cause the jaw member 325 to close.

FIG. 4 illustrates cutting tool 300 with jaw 325 in a closed position. While teeth 327 of jaw 325 are shown as forming an over-bite relative to teeth 320 of cutting assembly 315, such an arrangement is not necessary. For example, jaw member 325 and teeth 327 may form an under-bite relative to teeth 320 of cutting assembly 315, or may cooperate therewith to form a tight and sealed enclosure. Other arrangements are possible.

FIGS. 4a and 4b illustrate embodiments of tool arrangements that result in the described operation, namely the relative lateral movement of outer member 205 upon actuation of the trigger. FIG. 3a transfers the force created by squeezing trigger 360 to outer member 304 via L-shape link 355, which may be attached to or comprise a part of outer member 305. During actuation of trigger 360, pivoting of the interconnection members may occur at hinges 370 and 375. Inner tubular member 310 may be attached to or comprise a part of handle member 365, as shown. FIG. 4b illustrates a variation of FIG. 3a that works in the same way. Other arrangements are of course possible.

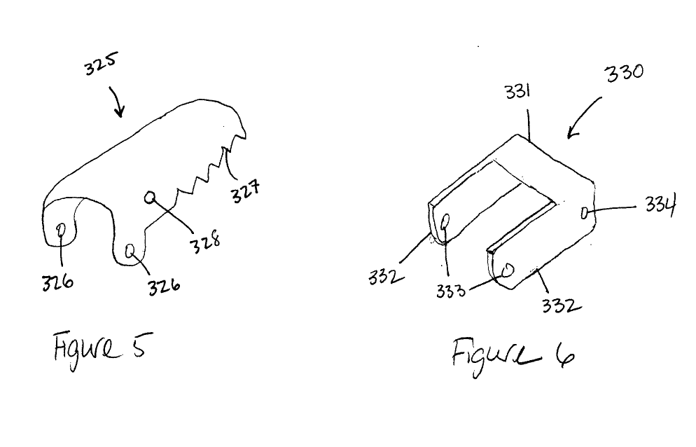

FIG. 5 illustrates one embodiment of the jaw member 325 of tool 300. As shown, jaw 325 comprises teeth 327 In some embodiments, jaw member 325 may comprise a smooth or grooved surface rather than teeth. Also shown are pin holes 326 and 327 for attaching the jaw member to the inner tubular member 310 and link 330, respectively.

FIG. 6 illustrates one embodiment of a link 330 of tool 300. As shown, link 330 comprises a base 331, two arms 332 with corresponding pin holes 333 and 334. As shown in FIGS. 3 and 4, pin holes 333 are attached to outer tubular member 305 while pin hole 334 is attached to jaw member 325.

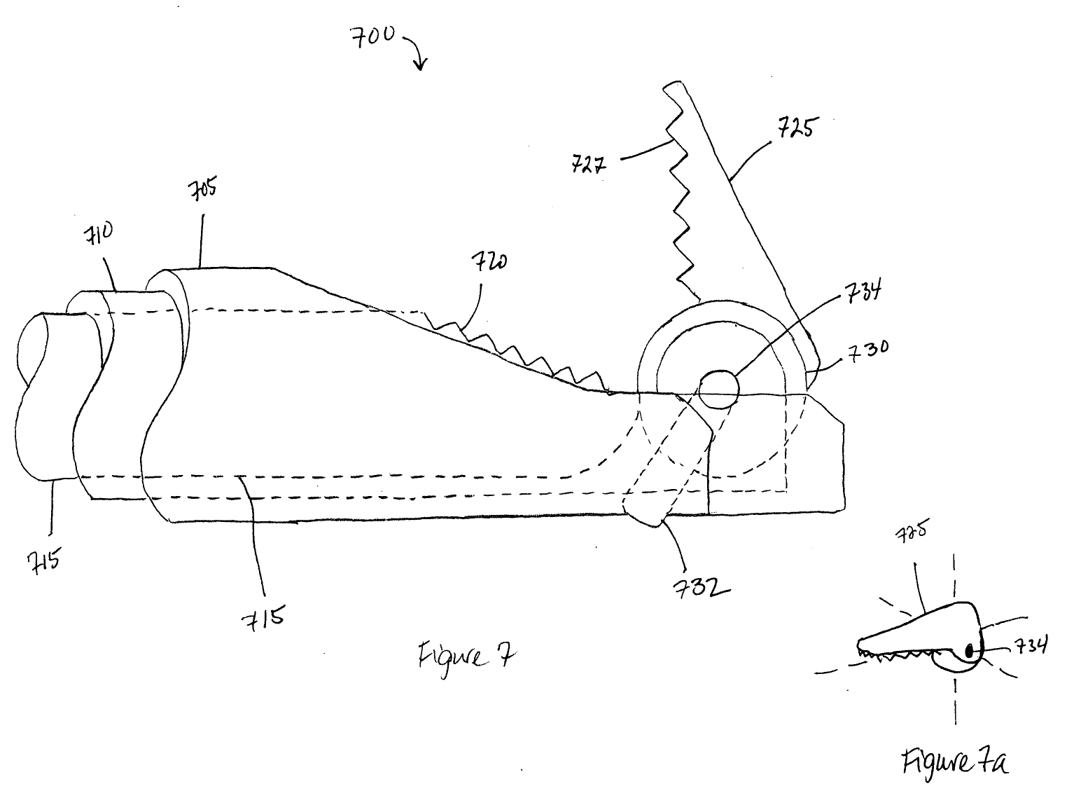

FIG. 7 illustrates a cutting tool 700 with an aft-grabbing jaw member, according to various embodiments of the systems and methods described herein. As shown, tool 700 comprises an outer tubular member 705, an inner tubular member 710 movably received within member 705, a jaw member 725 with teeth 727, a pivot ball 730 with pins 732 and 734, and a cutting assembly 715 with cutting portion 720. Preferably, outer and inner tubular members comprise rigid hollow and elongated tubes. In some embodiments, jaw member 725 is pivotally attached to inner tubular member 710 via pin 740. Jaw member 725 may also be pivotally attached to or pivot about outer member 705 via pin 732. Ball 730 may be received within jaw 725 to urge jaw member 325 to an open or closed position, depending on the relative positioning of outer and inner members 705 and 710. Ball 730 may also be pivotally attached to pins 732 and 734, as shown. FIG. 7a illustrates a perspective view of an interconnection between jaw 725 and ball 730, according to one embodiment. In this arrangement, actuating (e.g. squeezing) the trigger member (not shown) associated with tool 700 may result in outer member 705 moving in a lateral direction relative to inner tubular member 710. Thus, when the trigger is actuated, outer member 705 may move rightward relative to inner tubular member 310 (see FIGS. 4a and 4b for tool arrangements that apply the requisite forces). Such action results in a force being transferred to pin 732 that translates to a rotational force about pins 734 that causes jaw member 325 to open to the position as shown.

FIG. 8 illustrates cutting tool 300 with jaw 725 in a closed position. While teeth 727 of jaw 725 are shown as almost perfect fit with teeth 720 of cutting assembly 715, such an arrangement is not necessary. For example, jaw member 725 and teeth 727 may form an under-bite or over-bite relative to teeth 720 of cutting assembly 315, or may cooperate therewith to form a tight and sealed enclosure. Other arrangements are possible. FIG. 8a illustrates a cross-sectional view of the arrangement between outer and inner members 705 and 710, ball 730, and pins 732 and 734.

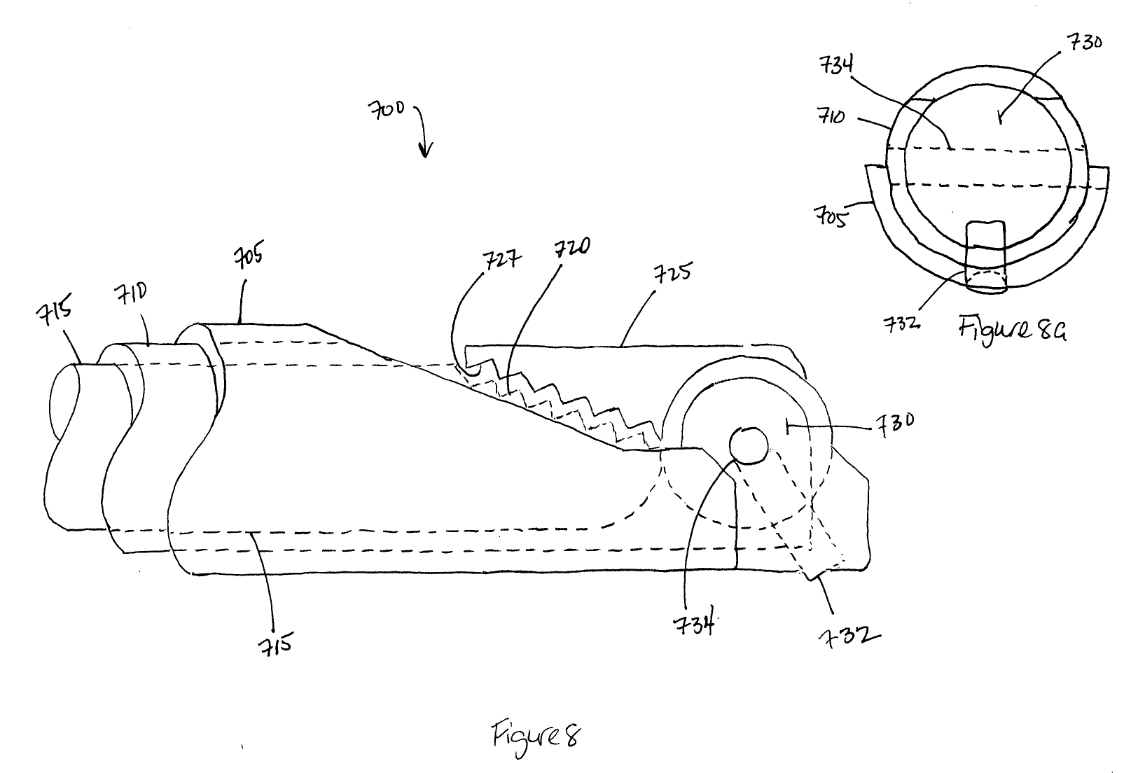

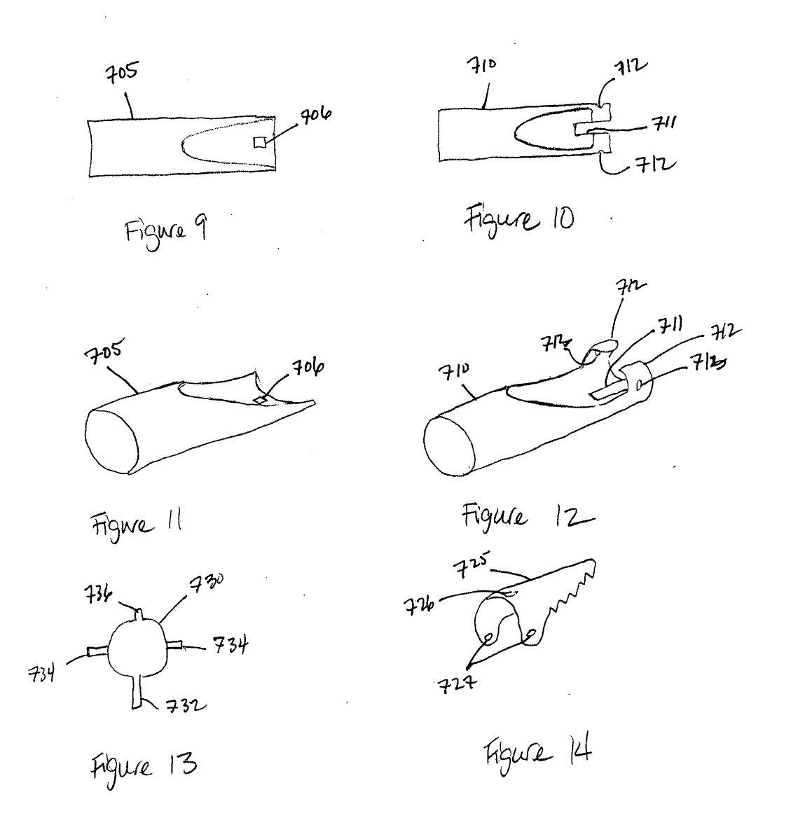

FIGS. 9 and 11 illustrate top and perspective views, respectively, of outer tubular member 705. As shown, outer member 705 may comprise a pivot window 706 that may receive pivot 732 to enable ball 730 to pivot about outer member 705 during operation. Preferably, the size of pivot window is such that pin 732 is able to smoothly transition between the positions shown in FIGS. 7 and 8.

FIGS. 10 and 12 illustrate top and perspective views, respectively, of inner tubular member 710. As shown, inner member 710 may comprise pivot window 711 and flanges 712 with pin holes 713. In some embodiments, window 711 accommodates pin 732 during actuation, while ball 730 rotates about pin holes 713.

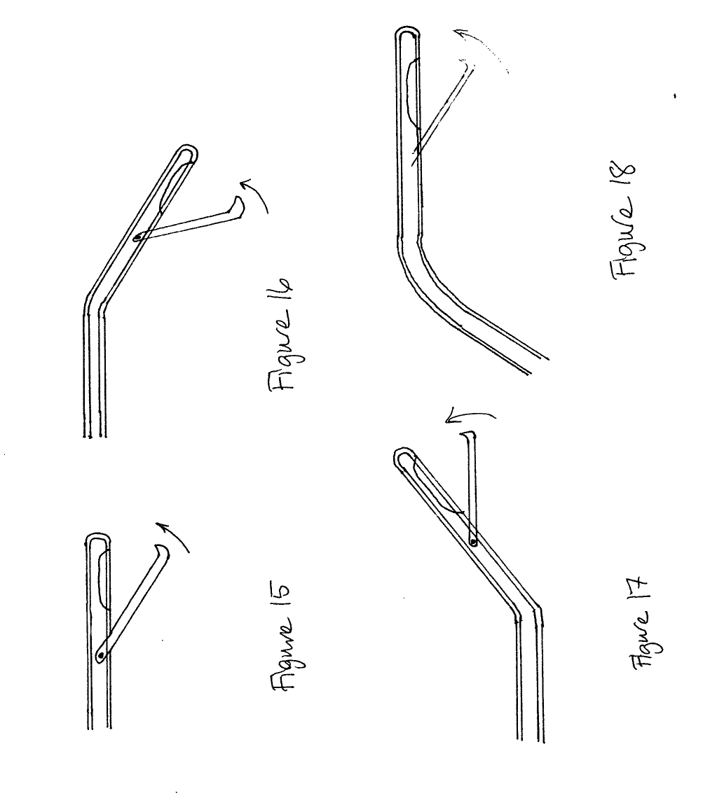

FIG. 13 illustrates one embodiment of ball 730 with associated pins 732, 734 and 736. As shown in FIGS. 7 and 8, pin 732 enables ball 730 to effectively pivot about window 706 of outer member 705. However, in so doing ball 703 is laterally stationary relative to inner member 710, although it rotates about pins 734. Pin 736 may also be used (although not used in FIGS. 7 and 8) to better urge jaw 725 to its appropriate position (e.g., opened or closed). In some embodiments, pin 736 may correspond to a pin hole associated with jaw 725.

FIG. 14 illustrates one embodiment of jaw 725 having teeth 727, and pin holes 726 and 728. As shown in FIGS. 7 and 8, pin holes 728 may enable jaw 725 to pivot about inner member 710 in the manner described.

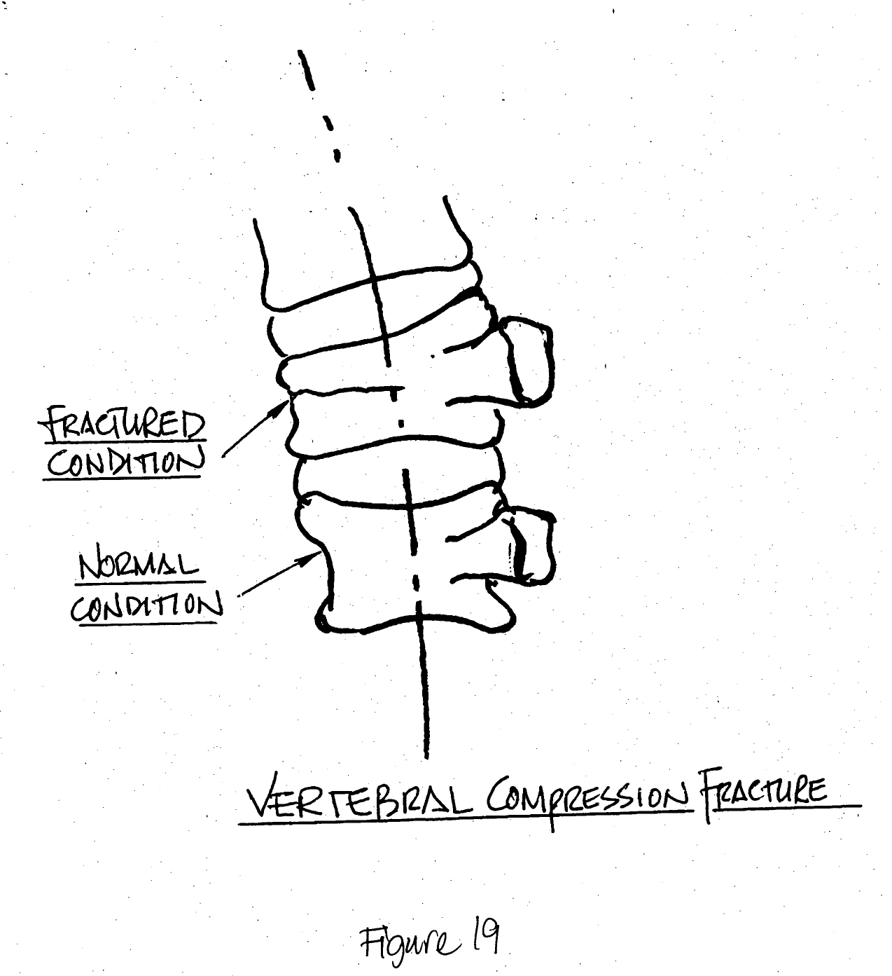

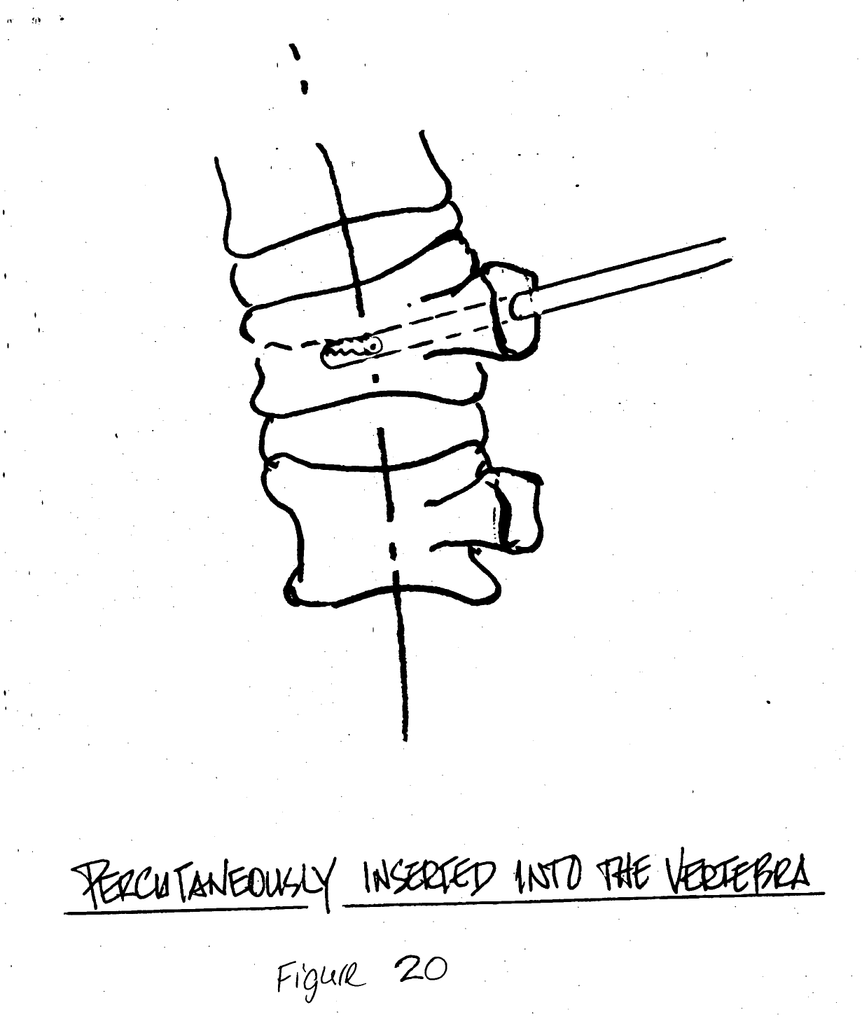

FIGS. 15-18 illustrate various embodiments of the cutting tools described herein. FIG. 15 depicts the straight tip embodiment described above and shown in FIGS. 1, 2, 2c, 3, 4, 7, and 8. FIG. 16 depicts a positive angled or curved tip. FIG. 17 illustrates a negative angled or curved tip. FIG. 18 illustrates a positive angled or curved tip with associated jaw closing backwards (e.g., an aft grabbing jaw).

The embodiments depicted in FIGS. 15-18 may comprise an upper jaw member rather than a lower jaw member as shown. FIGS. 3, 4, 7 and 8, for example, illustrate such an upper jaw implementation. In some embodiments, curved tip implementations may be realized by using a rigid outer member and an inner member that is flexible in the manner described herein and in referenced patents and applications. For example, such an inner member may be constructed in the manner described above and in FIGS. 2j and 2k. Other flexible constructions are possible, such as a single piece flexible tube, for example.

The various embodiments described are only exemplary and other embodiments are possible. In some embodiments, for example, the mechanical jaw or arm described above may be arranged to operate in a plane perpendicular to the position of the handle and trigger, in a plane parallel to the outer member, or within any angular position desired or necessary to the particular use employed, for example. The jaw or arm may also be arranged to be selectively moveable by a user within any defined range of movement. In some embodiments, the tissue cutting tool may comprise a continuous cutting assembly associated with or near a distal end of a cutter handle assembly, and a mechanical gathering assembly attached to or near the distal end of the cutter assembly. For example, any currently available rotary cutter may be modified to include a jaw element that is selectively actuated by a trigger assembly to: (1) grab tissue, particles, debris, or foreign material (2) cut tissue, particles, debris, or foreign material and/or (3) cooperate with the continuous cutting assembly to cut or remove tissue, particles, debris, or foreign material. Other embodiments are of course possible.

The instruments (and various components thereof) described herein may be made from a variety of materials, including, for example, medical plastics such polyvinyl chlorides, polypropylenes, polystyrenes, acetal copolymers, polyphenyl sulfones, polycarbonates, acrylics, silicone polymers, and mixtures and combinations thereof. Medical alloys such as titanium, titanium alloys, tantalum, tantalum alloys, stainless steel alloys, cobalt-based alloys, cobalt-chromium alloys, cobalt-chromium-molybdenum alloys, niobium alloys, and zirconium alloys also may be used to fabricate the instrument. Additionally, while many of the embodiments described herein relate to tubular inner and outer members, some embodiments may use different shapes (e.g., square) in constructing such members.

In another embodiment of the systems and methods described herein, methods for preparing an intervertebral space are provided, such as to prepare the intervertebral space to receive a prosthetic disc, a nucleus replacement implant, or a fusion device, for example. Instruments may be provided as described herein, for example, that comprise a cutting tool for aggressively and continuously cutting and/or removing tissue from an intervertebral space. Other exemplary surgical procedures are possible, such as that described in U.S. patent application Ser. No. 11/048,064, filed on Feb. 2, 2005, and titled “Method and Kit for Repairing a Defect in Bone,” the disclosure of which is incorporated herein by reference.

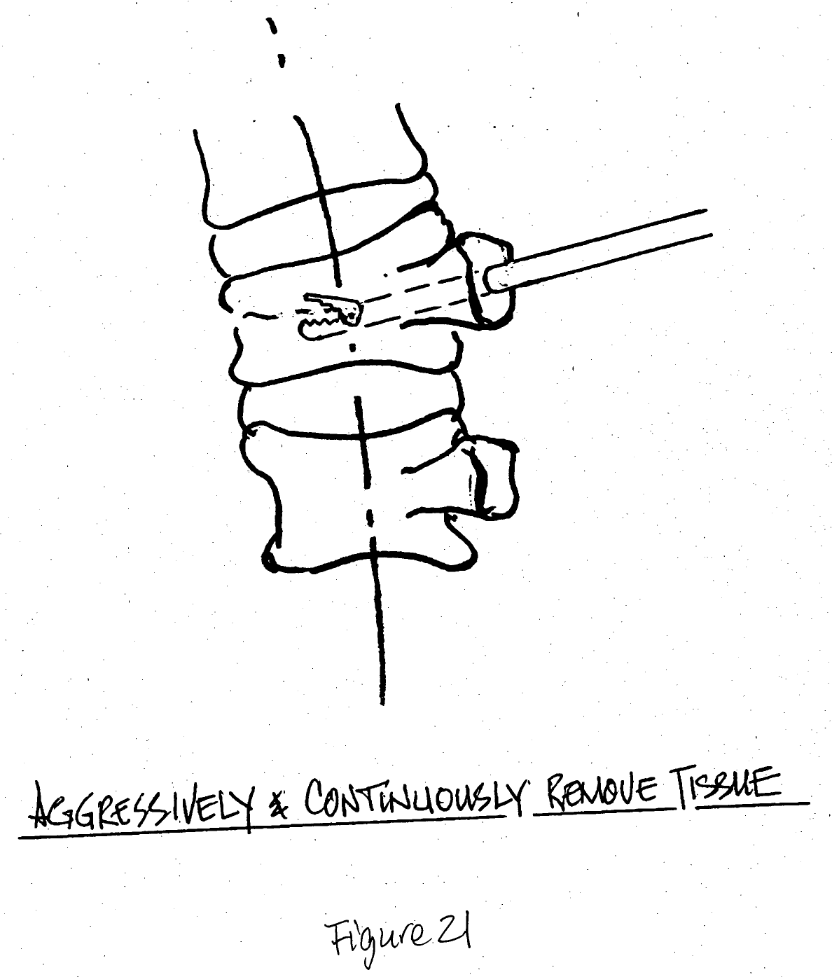

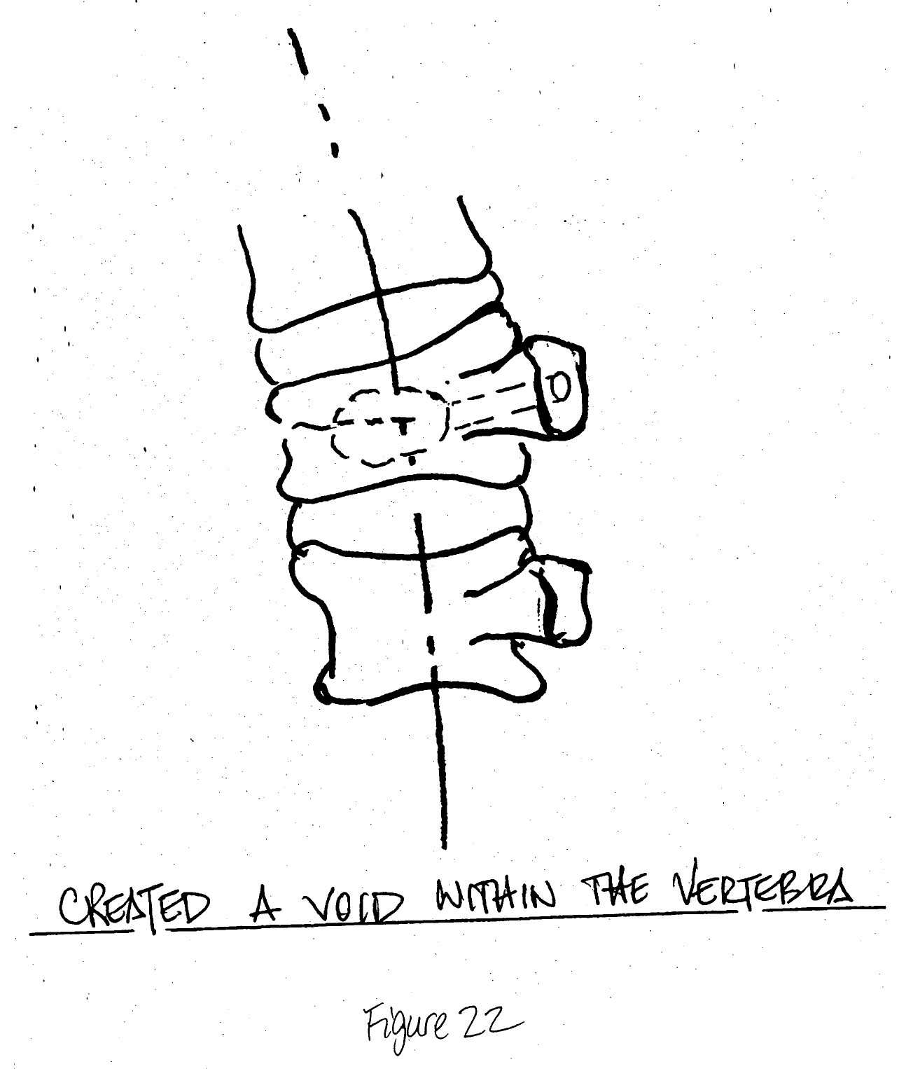

The systems and methods described herein may also be used to perform vertebroplasty and kyphoplasty surgical procedures. Vertebroplasty comprises a procedure wherein a path is created through the pedicle of a vertebra to access a fracture within the vertebra, as shown in FIG. 19. Typically, such a procedure is currently done by pushing a trocar or needle through a hole in the pedicle to forcibly displace tissue and create a path. The various systems and methods described herein, however, may be used to aggressively and continuously cut and remove tissue without using a trocar or needle, as shown in FIGS. 20-21, for example. (FIG. 20 shows the jaw member in a closed position).

Once the path is formed, the systems and methods described herein may also be used to perform a kyphoplasty procedure. Such a procedure comprises creating a void within the vertebra into which an implant or bio-material, such as polymethylmethacrylate or calcium phosphate bone cement, for example, may be inserted. Typically, such a void is currently created by inserting a balloon-like device within the path that is subsequently inflated to create a void/space by displacing tissue. The various systems and methods described, however, may be used to create the void by aggressively and continuously cutting and removing tissue without the balloon-like device, as shown in FIG. 22, for example.

In some embodiments, the systems and methods described herein may be used along with various other technology(ies), known or subsequently developed, that may facilitate the cutting and removal process, such as, for example, mechanical, electrical, high-pressure water jet cutting, laser, cryo (freezing), thermal, ultrasonic, and radio-frequency technologies. Other technologies are possible.

The foregoing detailed description is provided to describe the systems and methods described herein in detail, and is not intended to limit the various systems and methods. Those skilled in the art will appreciate that various modifications may be made to the systems and methods described herein without departing significantly from the spirit and scope thereof.

What is claimed is:

1. A tissue cutting tool, comprising:
an elongated outer tube having a proximal end and a distal end;
an elongated inner tube having a proximal end and a distal end, the elongated inner tube being moveably received in the elongated outer tube;
a cutting assembly being received in the elongated inner tube;
a handle arm attached near the proximal end of the outer tube;
a trigger arm attached near the proximal end of the inner tube; and
a movable jaw pivotally attached near the distal end of the outer or inner tube, the movable jaw being urged towards a cutting portion of the cutting blade assembly as the trigger arm is actuated.

2. The cutting tool of claim 1 wherein the movable jaw further comprises teeth formed along its periphery.

3. The cutting tool of claim 1 wherein the movable jaw comprises an aft grabbing movable jaw.

4. The cutting tool of claim 3 wherein the movable jaw is hinged to the inner tube and pivotally attached to the outer tube.

5. The cutting tool of claim 4 wherein the movable jaw is rigidly attached to the outer tube via a pivot pin.

6. The cutting tool of claim 1 wherein the movable jaw comprises a forward grabbing movable jaw.

7. The cutting tool of claim 6 wherein the movable jaw is directly hinged to the outer tube and hinged to the inner tube via a link element.

8. The cutting tool of claim 1 wherein the cutting assembly comprises:
an outer cutting blade having a cutting window near the distal end with teeth formed thereon;
an inner cutting blade rotatably received in the outer cutting blade, the inner cutting blade having a cutting window near the distal end with teeth formed thereon, the teeth of the inner cutting blade cooperating with the teeth of the outer cutting blade as the inner cutting blade is rotated, the inner cutting blade defining a cylindrical wall comprising a suction passage to permit aspiration of cut tissue through the cutting blade; and
power means for rotating the inner cutting blade within the outer cutting blade.

9. The cutting tool of claim 1 wherein the cutting blade assembly comprises:
an outer tubular member;
an inner tubular member rotatably received in the outer tubular member, the inner tubular member having a cutting member disposed at a distal end, the inner tubular member further comprising a suction passage to permit aspiration and removal of cut tissue through the inner tubular member;
aspiration means for aspirating and removing cut tissue through the inner tubular member; and
power means for rotating the inner tubular member and the shaver.

10. An anatomical tissue cutting tool, comprising:
a bent outer tube having a proximal end and a distal end;
a flexible inner tube having a proximal end and a distal end, the elongated inner tube being moveably received in the elongated outer tube;
a cutting blade assembly being received in the flexible inner tube;
a handle arm attached near the proximal end of the outer tube;
a trigger arm attached near the proximal end of the inner tube; and
a movable jaw pivotally attached near the distal end of the outer or inner tube, the movable jaw being urged towards a cutting portion of the cutting blade assembly as the trigger arm is actuated.

11. The cutting tool of claim 10 wherein the movable jaw further comprises teeth formed along its periphery.

12. The cutting tool of claim 10 wherein the movable jaw comprises an aft grabbing jaw.

13. The cutting tool of claim 10 wherein the movable jaw comprises a forward grabbing jaw.

14. The cutting tool of claim 10 wherein the outer tube is rigid.

15. A flexible inner member assembly for being moved in a bent outer member assembly, comprising:
a flexible member defining an inner lumen for receiving a cutting assembly, the flexible member having a jaw or arm member pivotally attached thereto;
a trigger member associated with the bent outer tubular member for actuating the jaw or arm member towards the cutting assembly.

16. The system of claim 15 wherein the flexible member comprises a tubular element containing at least one cut.

17. The system of claim 15 wherein the flexible member comprises a single-piece flexible tube.

18. The system of claim 15 wherein the jaw member comprises an aft-grabbing jaw.

19. The system of claim 15 wherein the jaw member comprises a forward-grabbing jaw.

20. The system of claim 15 wherein the bent outer member is rigid.

21. A tissue cutter, comprising:
a continuous cutting assembly associated with or near a distal end of a cutter handle assembly; and
a mechanical gathering assembly attached to or near the distal end of the cutter assembly.

22. The cutter of claim 21 wherein a proximal end of the cutter assembly is attached or connected to a power source for driving the continuous cutting assembly.

23. The cutter of claim 21 wherein the continuous cutting assembly operates to remove tissue from the cutting assembly.

24. The cutter of claim 21 wherein removal is further accomplished by a suction source attached or connected to or near the proximal end of the cutting assembly.

25. The cutting tool of claim 21 wherein the mechanical gathering assembly is selectively actuated by a trigger assembly to: (1) grab tissue, particles, debris, or foreign material, (2) cut tissue, particles, debris, or foreign material, or (3) cooperate with the continuous cutting assembly to cut and remove tissue, particles, debris or foreign material.

26. A method for cutting tissue, comprising:
entering an area containing tissue, particles, debris or foreign material with the cutter recited in claim 21; and
using the cutter to aggressively and continuously cut tissue, particles debris, or foreign material from within the area.

27. The method of claim 26 wherein area containing tissue comprises an intervertebral space.

28. The method of claim 26 wherein the area containing tissue comprises a vertebra.

29. The method of claim 26 further comprising the step of aggressively and continuously removing cut tissue from within the area.

30. The method of claim 29 wherein the cut tissue is removed from the area with the assistance of a suction source connected to the cutter.
